(19) United States
(12) Patent Application Publication
(10) Pub. No.: US 2014/0248365 A1
(43) Pub. Date: Sep. 4, 2014

(54) NANOPARTICLE PEPTIDE COMPOSITIONS

(71) Applicant: Midatech Limited, Abingdon (GB)

(72) Inventors: Thomas Rademacher, Oxfordshire (GB); Phillip Williams, Oxfordshire (GB)

(21) Appl. No.: 14/174,221

(22) Filed: Feb. 6, 2014

(30) Foreign Application Priority Data
Mar. 4, 2013 (GB) 1303771.8

(51) Int. Cl. A61K 9/14; A61K 38/29

(52) U.S. Cl. CPC A61K 9/146; A61K 38/29
USPC 424/491; 514/11.8

(57) ABSTRACT

The present invention relates to teriparatide peptide-carrying nanoparticles, particularly for use in medicine, and includes methods for treatment of disorders, e.g., of bone density. Nanoparticle composition comprise a nanoparticle comprising a core comprising a metal and/or a semiconductor; and a corona comprising a plurality of ligands covalently linked to the core, wherein said plurality of ligands comprise at least one glutathione; and at least one teriparatide peptide that is non-covalently bound to the corona.

### FIELD OF THE INVENTION

The present invention relates to peptide-carrying nanoparticles, particularly for use in medicine, and includes methods for treatment of disorders, e.g., of bone density.

### BACKGROUND TO THE INVENTION

The present invention is directed at compositions and products, and methods of making and administering such compositions and products, including for the treatment of mammals and particularly humans.

Bioactive agents, such as peptides, frequently suffer from poor stability, particularly thermo-stability, which may limit the conditions to which the agents can be subjected during preparation, processing, storage and/or delivery. Medical preparations of peptides for human use are generally formulated with one or more preservatives and/or stabilisers. Moreover, limited gastrointestinal stability typically presents a barrier to effective oral administration of bioactive peptides.

WO 2011/154711 describes glyconanoparticles that have a gold core surrounded by a carbohydrate corona and which act as carriers for peptides such as insulin.

Teriparatide, a recombinant fragment (residues 1-34) of human parathyroid hormone, is used in osteoporosis therapy typically via daily subcutaneous (s.c.) injection.

There remains an unmet need for further nanoparticle compositions capable of carrying bioactive peptides, and for methods of delivering such bioactive peptides to a subject.

The present invention addresses these and other needs.

### BRIEF DESCRIPTION OF THE INVENTION

Broadly, the present invention relates to teriparatide peptide-carrying nanoparticle compositions. The present inventors have found that nanoparticles having a corona of glutathione ligands bind teriparatide (in some cases with a binding capacity of around 15 teriparatide molecules per nanoparticle). Nanoparticles as defined herein therefor provide a carrier for the formulation and delivery of teriparatide to subjects in need of teriparatide therapeutic treatment.

Accordingly, in a first aspect the present invention provides a nanoparticle composition comprising:
(a) a nanoparticle comprising:
   (i) a core comprising a metal and/or a semiconductor;
   (ii) a corona comprising a plurality of ligands covalently linked to the core, wherein said plurality of ligands comprise at least one glutathione; and
(b) at least one teriparatide peptide that is non-covalently bound to the corona.

In accordance with any one of the aspects of the present invention, the teriparatide peptide may comprise or consist of an amino acid sequence having at least 70%, 80%, 90%, 95% or 99% amino acid sequence identity with the full-length amino acid sequence set forth as SEQ ID NO: 1. In some cases, the teriparatide peptide comprises or consists of the full-length amino acid sequence SVSEIQLMHNLGKHLNSMERVEWLRKKLQDVHNF (SEQ ID NO: 1).

SEQ ID NO: 1 is the 34 amino acid sequence of residues 32-65 of the complete 115 amino acid sequence of the human parathyroid hormone polypeptide set forth below as SEQ ID NO: 2 and disclosed under UniProt accession no. P01270, version 136, dated 31 Oct. 2012.

>sp|P01270|PTHY_HUMAN Parathyroid hormone OS=Homo sapiens GN=PTH PE=1 SV=1

MIPAKDMAKVMIVMLAICFLTKSDGKSVKKRSVSEIQLMHNLGKHLNSMERVEWLRKKLQDVHNFVALGAPLAPR DAGSQRPRKKEDNVLVESHEKSLGEADKADVNVLTKAKSQ (SEQ ID NO: 2). The 84 amino acid sequence of the mature human parathyroid hormone (residues 32-115) is shown in italics (SEQ ID NO: 3). The 34 amino acid sequence of teriparatide (residues 32-65) is shown underlined (SEQ ID NO: 1).

>sp|P01270|32-115
(SEQ ID NO: 3)
SVSEIQLMHNLGKHLNSMERVEWLRKKLQDVHNFVALGAPLAPRDAGSQRPRKKEDNVLVESHEKSLG
EADKADVNVLTKAKSQ.

In certain cases in accordance with the present invention, the teriparatide peptide may be selected from the group consisting of:
(i) a peptide comprising or consisting of an amino acid sequence having at least 70%, 80%, 90%, 95% or 99% amino acid sequence identity to the full-length sequence set forth in SEQ ID NO: 1 or 3;
(ii) a peptide comprising or consisting of the full-length amino acid sequence set forth in SEQ ID NO: 1 or 3;
(iii) a peptide comprising or consisting of a variant sequence of the full-length amino acid sequence set forth in SEQ ID NO: 1 or 3, wherein said variant differs by addition, deletion, substitution or modification of not more than 1, 2, 3, 4, 5, 6, 7, 8, 9 or not more than 10 amino acids from said full-length amino acid sequence set forth in SEQ ID NO: 1 or 3;
(iv) a peptide comprising or consisting of a contiguous fragment of any one of (i)-(iii), said fragment having a sequence length of at least 15, 20, 25 or 30 amino acids.

Preferably, the teriparatide peptide exhibits biological activity of teriparatide. In particular, said teriparatide peptide of any one of (i)-(iv) may exhibit at least 50% of the activity of the teriparatide peptide of SEQ ID NO: 1 or at least 50% of the activity of the peptide of SEQ ID NO: 3 in an in vitro or in vivo bioassay of teriparatide activity. In certain cases, the teriparatide activity may comprise one or more activities selected from the group consisting of: PTH receptor agonist activity; modification of the osteoblast/osteoclast bone formation/resorption balance; enhancement of kidney calcium and/or magnesium reabsorption; regulation of plasma calcium and/or phosphate concentration; and enhancement of intestinal calcium absorption.

Parathyroid hormone (PTH) increases serum calcium, partially accomplishing this by increasing bone resorption. Thus, chronically elevated PTH will deplete bone stores. However, intermittent exposure to PTH has been found to activate osteoblasts and have an anabolic effect. The mechanism of this anabolic effect is unknown but clinical studies have confirmed that treatment with teriparatide such as once-daily injections of teriparatide, has the net effect of stimulating new bone formation leading to increased bone mineral density and improves bone mineral density and bone mineral content in patients with osteoporosis (Teriparatide: A Review Elaena Quattrocchi, PharmD, and Helen Kourlas, PharmD; Clin Ther. 2004:26:841-834). In preferred cases in accordance with any one of the aspects of the present invention, the teriparatide peptide exhibits the ability, e.g. upon intermittent administration to a mammalian subject, to produce a net positive effect on bone formation in the subject.

It has been found that the nanoparticles in accordance with the present invention may be provided with a variety of numbers of ligands forming the corona. For example, in some cases the corona comprises at least 5, 10, 20 or at least 50 ligands per core, e.g. between about 10 to about 1000 ligands per core. In particular, the nanoparticle compositions in accordance with any aspect of the present invention may comprise at least 5, 10, 15, 20 or at least 50 glutathione ligands per core.

The number of teriparatide peptide molecules bound per core is not particularly limited. For certain applications, it may be desirable to employ as few as 1, 2, 3 or 4 teriparatide peptides per core, while in other cases the nanoparticle of the invention may comprise at least 5, 10, 15, 20 or at least 50 or more teriparatide peptide molecules bound per core.

In some cases, in accordance with any one of the aspects of the present invention, the at least one teriparatide peptide may be bound to the corona of the nanoparticle in a reversible manner. In particular, the teriparatide peptide may be bound to the corona such that at least a fraction of the bound teriparatide peptide is released from the nanoparticle upon contacting the nanoparticle with a physiological solution.

In some cases, in accordance with any one of the aspects of the present invention, said ligands comprise glutathione alone or in conjunction with other species of ligand, e.g., combinations of glutathione and carbohydrate ligands (including glucose-containing ligands) are specifically contemplated herein.

In some cases, in accordance with any one of the aspects of the present invention, the nanoparticle comprises at least 10, at least 20, at least 30, at least 40 or at least 50 ligands which are (i) glutathione ligands; or (ii) both glutathione ligands and ligands other than glutathione, such as carbohydrate-containing ligands.

In some cases, in accordance with any one of the aspects of the present invention, the diameter of the core of the nanoparticle is in the range 1 nm to 5 nm.

In some cases, in accordance with any one of the aspects of the present invention, the diameter of the nanoparticle including its ligands is in the range 2 nm to 50 nm, optionally 3 nm to 30 nm, or 4 nm to 20 nm, or 5 nm to 15 nm.

In some cases, in accordance with any one of the aspects of the present invention, the core comprises a metal selected from the group consisting of: Au, Ag, Cu, Pt, Pd, Fe, Co, Gd and Zn, or any combination thereof.

In some cases, in accordance with any one of the aspects of the present invention, the core is magnetic.

In some cases, in accordance with any one of the aspects of the present invention, the core comprises a semiconductor. The semiconductor may comprise metal atoms, such as cadmium.

Alternatively or additionally, the semiconductor may comprise non-metal atoms. Organic semiconductors are specifically contemplated herein. Preferred semiconductors, in accordance with the present invention, may be selected from the group consisting of: cadmium selenide, cadmium sulphide, cadmium tellurium and zinc sulphide.

In some cases, in accordance with any one of the aspects of the present invention, the core is capable of acting as a quantum dot.

Preferably, the composition in accordance with the first aspect of the invention comprises a plurality, e.g., 100, 1000, 100000, or more, of said nanoparticles, wherein at least 10%, 20%, 30%, 40%, 50%, 60%, 70%, 80%, 90% or 95% of the nanoparticles in said composition have at least one teriparatide peptide bound.

In some cases, in accordance with any one of the aspects of the present invention, the nanoparticle composition comprises a carrier, such as solution, a polymer, a powder, or a cream, in which the nanoparticles and bound teriparatide peptides are suspended. In certain cases, the composition may be in the form of a patch or film for delivery to or across skin, mouth, vagina, rectum or in the form of a spray for delivery into the mouth, nose, lungs or the rectum or vagina. The composition may be in an associated form, a suspension or contained together in a single package, container or carrier. In certain cases, the composition may take the form of one or more doses (e.g. a defined quantity of teriparatide peptide or teriparatide peptide activity units), such as in the form of a therapeutic dose or defined number of doses.

In some cases, in accordance with any one of the aspects of the present invention, the nanoparticle composition further comprises at least one permeation enhancer that is non-covalently or covalently bound to said core and/or or said corona. As described in co-pending GB patent application No. 1301991.4, filed 5 Feb. 2013, the entire contents of which are expressly incorporated herein by reference for all purposes, certain permeation enhancers may be advantageously bound to the nanoparticle without displacing any significant active peptide, such as the amylin peptide as defined herein. In certain cases, said permeation enhancer is selected from an alkyl-D-maltoside (e.g. tetradecyl-D-maltoside, dodecyl-β-D-maltoside, hexyl-β-D-maltoside, octyl-β-D-maltoside, nonyl-β-D-maltoside, decyl-β-D-maltoside, undecyl-β-D-maltoside, tridecyl-β-D-maltoside, or hexadecyl-β-D-maltoside) and lysalbinic acid. In certain cases, said permeation enhancer, e.g. tetradecyl-D-maltoside, dodecyl-β-D-maltoside and/or lysalbinic acid is non-covalently bound to said corona.

In a second aspect, the present invention provides a nanoparticle composition as defined in accordance with the first aspect, for use in medicine.

In a third aspect, the present invention provides a nanoparticle composition as defined in accordance with the first aspect, for use in a method of therapeutic or prophylactic treatment of osteoporosis in a mammalian subject.

In a fourth aspect, the present invention provides use of a nanoparticle composition as defined in accordance with the first aspect in the preparation of a medicament for therapeutic or prophylactic treatment of osteoporosis in a mammalian subject.

In a fifth aspect, the present invention provides a method of therapeutic or prophylactic treatment of osteoporosis in a mammalian subject, the method comprising administering a therapeutically or prophylactically effective amount of a nanoparticle composition as defined in accordance with the first aspect to the subject in need of said treatment.

In a sixth aspect, the present invention provides a method of increasing bone mineral density in a mammalian subject, the method comprising administering an effective amount of a nanoparticle composition as defined in accordance with the first aspect to the subject.

In accordance with any one of the second to sixth aspects of the invention, the subject may be a human, a companion animal (e.g. a dog or cat), a laboratory animal (e.g. a mouse, rat, rabbit, pig or non-human primate), a domestic or farm animal (e.g. a pig, cow, horse or sheep). Preferably, the subject is a human. In some cases the subject is female, e.g. a human female such as a post-menopausal woman.

In accordance with any one of the second to sixth aspects of the invention, the subject may in certain cases have a disorder that results in abnormally lowered bone mineral density (e.g. lower than normal considering the subject's age and/or gender). In particular, specifically contemplated herein is a subjects having, or being at risk of developing, osteoporosis. The subject may or may not have previously been diagnosed with osteoporosis. For example, the subject may have been identified as being at risk of developing osteoporosis (e.g. by virtue of the subject's gender, age, environmental risk factors, medication history and/or presence of one or more biomarker risk factors). The subject may, in some cases, be following a course of treatment for osteoporosis. In particular, the subject may be taking, or have been advised to take, teriparatide, a bisphosphonate medication, hormone replacement therapy, calcium, vitamin D and/or vitamin K.

In accordance with any one of the second to sixth aspects of the invention, the nanoparticle composition may be administered or for administration with (i.e. simultaneously, separately or sequentially) one or more therapeutic agents for the control of bone mineral density, for example, a bisphosphonate medication, hormone replacement therapy, calcium, vitamin D, menatetrenone and/or vitamin K.

In accordance with any one of the second to sixth aspects of the invention, the nanoparticle composition may be administered or for administration by any suitable route. In particular cases, the nanoparticle composition may be administered or for administration via a route selected from the group consisting of: intravenous (i.v.), intramuscular (i.m.), intradermal (i.d.), intraperitoneal or subcutaneous (s.c.) injection or infusion; buccal; sublabial; sublingual; by inhalation; via one or more mucosal membranes; urogenital; rectal; intranasal and dermal.

In a seventh aspect, the present invention provides an article of manufacture comprising:
a nanoparticle composition as defined in accordance with the first aspect of the invention;
a container for housing the nanoparticle composition; and
an insert and/or label. Preferably, the insert and/or label provide instructions, dosage and/or administration information relating to the use of the nanoparticle composition in a method of treatment of a disorder of bone density. In particular, the disorder may be osteoporosis.

In an eighth aspect, the present invention provides a process for producing a nanoparticle composition as defined in accordance with the first aspect of the invention, the process comprising:
providing a nanoparticle comprising a core comprising a metal and/or a semiconductor and a corona comprising a plurality of ligands covalently linked to the core, wherein said ligands comprise glutathione; and
contacting the nanoparticle with at least one teriparatide peptide under conditions which allow the at least one teriparatide peptide to bind to the corona of the nanoparticle.

In some cases, in accordance with this aspect of the present invention, the process comprises an earlier step of producing the nanoparticle, said earlier step comprising: combining a solution comprising glutathione with a solution comprising a core-forming material (e.g. gold III chloride) and with a reducing agent (e.g. sodium borohydride), thereby causing the nanoparticle to self-assemble.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows Forteo (teriparatide) binding under varying GSH NP concentrations. The apparent downward trend of GSH NP may be explained by higher interference of the NPs on the BCA assay. Unlike GSH NP Zn, GSH NP showed apparent lower NP Forteo binding.

FIG. 2 shows the near identical binding capacity of GSHNP for Forteo (teriparatide) regardless of the presence of zinc ions, when corrected for actual NP in the pellet.

FIG. 3 shows Forteo (teriparatide) binding to variable ratio C2-Glucose and glutathione (GSH) ligand NPs.

FIG. 4 shows a binding curve with variable/excess GSHNP and a lower level of Forteo (teriparatide).

FIG. 5 shows a variable pH binding curve of Forteo (teriparatide) to GSHNPs.

### DETAILED DESCRIPTION OF THE INVENTION

In describing the present invention, the following terms will be employed, and are intended to be defined as indicated below.

As used herein, “nanoparticle” refers to a particle having a nanomeric scale, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry.

Nanoparticles comprising a plurality of carbohydrate-containing ligands have been described in, for example, WO 2002/032404, WO 2004/108165, WO 2005/116226, WO 2006/037979, WO 2007/015105, WO 2007/122388, WO 2005/091704 (the entire contents of each of which is expressly incorporated herein by reference) and such nanoparticles may find use in accordance with the present invention. Moreover, gold-coated nanoparticles comprising a magnetic core of iron oxide ferrites (having the formula XFe₂O₄, where X=Fe, Mn or Co) functionalised with organic compounds (e.g. via a thiol-gold bond) are described in EP2305310 (the entire contents of which is expressly incorporated herein by reference) and are specifically contemplated for use as nanoparticles/nanoparticle cores in accordance with the present invention.

As used herein, “corona” refers to a layer or coating, which may partially or completely cover the exposed surface of the nanoparticle core. The corona includes a plurality of ligands which generally include at least one carbohydrate moiety, one surfactant moiety and/or one glutathione moiety. Thus, the corona may be considered to be an organic layer that surrounds or partially surrounds the metallic core. In certain embodiments the corona provides and/or participates in passivating the core of the nanoparticle. Thus, in certain cases the corona may include a sufficiently complete coating layer substantially to stabilise the semiconductor or metal-containing core. However, it is specifically contemplated herein that certain nanoparticles having cores, e.g., that include a metal oxide-containing inner core coated with a noble metal may include a corona that only partially coats the core surface. In certain cases the corona facilitates solubility, such as water solubility, of the nanoparticles of the present invention.

Nanoparticles

Nanoparticles are small particles, e.g. clusters of metal or semiconductor atoms, that can be used as a substrate for immobilising ligands.

Preferably, the nanoparticles have cores having mean diameters between 0.5 and 50 nm, more preferably between 0.5 and 10 nm, more preferably between 0.5 and 5 nm, more preferably between 0.5 and 3 nm and still more preferably between 0.5 and 2.5 nm. When the ligands are considered in addition to the cores, preferably the overall mean diameter of the particles is between 2.0 and 20 nm, more preferably between 3 and 10 nm and most preferably between 4 and 5 nm. The mean diameter can be measured using techniques well known in the art such as transmission electron microscopy.

The core material can be a metal and/or semiconductor (said semiconductor optionally comprising metal atoms or being an organic semiconductor) and may be formed of more than one type of atom. Preferably, the core material is a metal selected from Au, Fe or Cu. Nanoparticle cores may also be formed from alloys including Au/Fe, Au/Cu, Au/Gd, Au/Fe/Cu, Au/Fe/Gd and Au/Fe/Cu/Gd, and may be used in the present invention. Preferred core materials are Au and Fe, with the most preferred material being Au. The cores of the nanoparticles preferably comprise between about 100 and 500 atoms (e.g. gold atoms) to provide core diameters in the nanometre range. Other particularly useful core materials are doped with one or more atoms that are NMR active, allowing the nanoparticles to be detected using NMR, both in vitro and in vivo. Examples of NMR active atoms include Mn⁺², Gd⁺³, Eu⁺², Cu⁺², V⁺², Co⁺², Ni⁺², Fe⁺², Fe⁺³ and lanthanides⁺³, or the quantum dots described elsewhere in this application.

Nanoparticle cores comprising semiconductor compounds can be detected as nanometre scale semiconductor crystals are capable of acting as quantum dots, that is they can absorb light thereby exciting electrons in the materials to higher energy levels, subsequently releasing photons of light at frequencies characteristic of the material. An example of a semiconductor core material is cadmium selenide, cadmium sulphide, cadmium tellurium. Also included are the zinc compounds such as zinc sulphide.

In some embodiments, the core of the nanoparticles may be magnetic and comprise magnetic metal atoms, optionally in combination with passive metal atoms. By way of example, the passive metal may be gold, platinum, silver or copper, and the magnetic metal may be iron or gadolinium. In preferred embodiments, the passive metal is gold and the magnetic metal is iron. In this case, conveniently the ratio of passive metal atoms to magnetic metal atoms in the core is between about 5:0.1 and about 2:5. More preferably, the ratio is between about 5:0.1 and about 5:1. As used herein, the term “passive metals” refers to metals which do not show magnetic properties and are chemically stable to oxidation. The passive metals may be diamagnetic or superparamagnetic. Preferably, such nanoparticles are superparamagnetic.

Examples of nanoparticles which have cores comprising a paramagnetic metal, include those comprising Mn⁺², Gd⁺³, Eu⁺², Cu⁺², V⁺², Co⁺², Ni⁺², Fe⁺², Fe⁺³ and lanthanides⁺³.

Other magnetic nanoparticles may be formed from materials such as MnFe (spinel ferrite) or CoFe (cobalt ferrite) can be formed into nanoparticles (magnetic fluid, with or without the addition of a further core material as defined above. Examples of the self-assembly attachment chemistry for producing such nanoparticles is given in Biotechnol. Prog., 19:1095-100 (2003), J. Am. Chem. Soc. 125:9828-33 (2003), J. Colloid Interface Sci. 255:293-8 (2002).

In some embodiments, the nanoparticle or its ligand comprises a detectable label. The label may be an element of the core of the nanoparticle or the ligand. The label may be detectable because of an intrinsic property of that element of the nanoparticle or by being linked, conjugated or associated with a further moiety that is detectable. Preferred examples of labels include a label which is a fluorescent group, a radionuclide, a magnetic label or a dye. Fluorescent groups include fluorescein, rhodamine or tetramethyl rhodamine, Texas-Red, Cy3, Cy5, etc., and may be detected by excitation of the fluorescent label and detection of the emitted light using Raman scattering spectroscopy (Y. C. Cao, R. Jin, C. A. Mirkin, Science 2002, 297: 1536-1539).

In some embodiments, the nanoparticles may comprise a radionuclide for use in detecting the nanoparticle using the radioactivity emitted by the radionuclide, e.g. by using PET, SPECT, or for therapy, i.e. for killing target cells. Examples of radionuclides commonly used in the art that could be readily adapted for use in the present invention include ^{99m}Tc, which exists in a variety of oxidation states although the most stable is TcO⁴⁻; ³²P or ³³P; ⁵⁷Co; ⁵⁹Fe; ⁶⁷Cu which is often used as Cu²⁺ salts; ⁶⁷Ga which is commonly used a Ga³⁺ salt, e.g. gallium citrate; ⁶⁸Ge; ⁸²Sr; ⁹⁹Mo; ¹⁰³Pd; ¹¹¹In which is generally used as In³⁺ salts; ¹²⁵I or ¹³¹I which is generally used as sodium iodide; ¹³⁷Cs; ¹⁵³Gd; ¹⁵³Sm; ¹⁵⁸Au; ¹⁸⁶Re; ²⁰¹Tl generally used as a Tl⁺ salt such as thallium chloride; ³⁹Y³⁺; ⁷¹Lu³⁺; and ²⁴Cr²⁺. The general use of radionuclides as labels and tracers is well known in the art and could readily be adapted by the skilled person for use in the aspects of the present invention. The radionuclides may be employed most easily by doping the cores of the nanoparticles or including them as labels present as part of ligands immobilised on the nanoparticles.

Additionally or alternatively, the nanoparticles of the present invention, or the results of their interactions with other species, can be detected using a number of techniques well known in the art using a label associated with the nanoparticle as indicated above or by employing a property of them. These methods of detecting nanoparticles can range from detecting the aggregation that results when the nanoparticles bind to another species, e.g. by simple visual inspection or by using light scattering (transmittance of a solution containing the nanoparticles), to using sophisticated techniques such as transmission electron microscopy (TEM) or atomic force microscopy (AFM) to visualise the nanoparticles. A further method of detecting metal particles is to employ plasmon resonance that is the excitation of electrons at the surface of a metal, usually caused by optical radiation. The phenomenon of surface plasmon resonance (SPR) exists at the interface of a metal (such as Ag or Au) and a dielectric material such as air or water. As changes in SPR occur as analytes bind to the ligand immobilised on the surface of a nanoparticle changing the refractive index of the interface. A further advantage of SPR is that it can be used to monitor real time interactions. As mentioned above, if the nanoparticles include or are doped with atoms which are NMR active, then this technique can be used to detect the particles, both in vitro or in vivo, using techniques well known in the art. Nanoparticles can also be detected using a system based on quantitative signal amplification using the nanoparticle-promoted reduction of silver (I). Fluorescence spectroscopy can be used if the nanoparticles include ligands as fluorescent probes. Also, isotopic labelling of the carbohydrate can be used to facilitate their detection.

### Teriparatide Peptide

In certain cases in accordance with the present invention, the “teriparatide peptide” may be selected from the group consisting of:
(i) a peptide comprising or consisting of an amino acid sequence having at least 70%, 80%, 90%, 95% or 99% amino acid sequence identity to the full-length sequence set forth in SEQ ID NO: 1 or 3;
(ii) a peptide comprising or consisting of the full-length amino acid sequence set forth in SEQ ID NO: 1 or 3;
(iii) a peptide comprising or consisting of a variant sequence of the full-length amino acid sequence set forth in SEQ ID NO: 1 or 3, wherein said variant differs by addition, deletion, substitution or modification of not more than 1, 2, 3, 4, 5, 6, 7, 8, 9 or not more than 10 amino acids from said full-length amino acid sequence set forth in SEQ ID NO: 1 or 3;
(iv) a peptide comprising or consisting of a fragment of any one of (i)-(iii), said fragment having a sequence length of at least 15, 20, 25 or 30 amino acids.

Sequence identity may be calculated using any suitable method, as would be readily apparent to the skilled person. In certain cases, amino acid sequence identity between a candidate sequence and a reference sequence, e.g. the sequence of SEQ ID NO: 1, may be calculated using the online tool SUPERMATCHER available at the following URL: http://emboss.bioinformatics.nl/cgi-bin/emboss/supermatcher using GAP opening penalty of 10.0 and GAP extension penalty of 0.5 (see EMBOSS: The European Molecular Biology Open Software Suite (2000) Rice, P. Longden, I. and Bleasby, A. Trends in Genetics 16, (6) pp. 276-277).

Preferably, said teriparatide peptide of any one of (i)-(iv) exhibits biological activity of teriparatide. In particular, said teriparatide peptide of any one of (i)-(iv) may exhibit at least 50% of the activity of the teriparatide peptide of SEQ ID NO: 1 or at least 50% of the activity of the teriparatide peptide of SEQ ID NO: 3 in an in vitro or in vivo bioassay of teriparatide activity. In certain cases, the teriparatide activity may comprise PTH receptor agonist activity; modification of the osteoblast/osteoclast bone formation/resorption balance; enhancement of kidney calcium and/or magnesium reabsorption; regulation of plasma calcium and/or phosphate concentration; enhancement of conversion of 25(OH) D3 to 1, 25(OH)₂ vitamin D3; and/or enhancement of intestinal calcium absorption. As used herein, “teriparatide” and “Forteo”® are used interchangeably.

The teriparatide peptide is bound to the corona of the nanoparticle. Without wishing to be bound by any theory, it is presently believed that the teriparatide peptide may participate in one or more reversible binding interactions with one or more ligands that provide the corona of the nanoparticle. In particular, a portion of the sequence of amino acids may participate in hydrogen bonding, Van der Waals forces and/or electrostatic interactions with one or more ligands (e.g. interacting with one or more glutathione ligands). The peptide binding may involve adsorption, absorption or other direct or indirect interaction with one or more ligands of the nanoparticle.

As described herein with reference to certain embodiments of the present invention, the teriparatide peptide may be bound such that at least a fraction or portion of the bound teriparatide peptide is released from the nanoparticle upon contacting the nanoparticle with a physiological solution. As described herein the teriparatide peptide may be bound to the nanoparticle in a manner such that the teriparatide peptide is stabilised (e.g. thermostabilised) while bound, but is releasable and available in a form that is biologically active (for example, releasable such that the teriparatide peptide is detectable by ELISA and/or capable of exerting at least one biological action in an in vitro or in vivo assay system that is characteristic of the free teriparatide peptide). In particular, the teriparatide peptide may be bound to the nanoparticle such that a suspension of the teriparatide-bound nanoparticles gives a positive result in an ELISA for, e.g., (human) teriparatide and/or exerts an effect on a PTH receptor (e.g. expressed on a cell surface) and/or exerts an effect on bone mineral density in a mammalian subject.

### Administration and Treatment

The nanoparticles and compositions of the invention may be administered to patients by any number of different routes, including enteral or parenteral routes. Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), film, patch and rectal systemic routes.

Administration be performed e.g. by injection, or ballistically using a delivery gun to accelerate their transdermal passage through the outer layer of the epidermis. The nanoparticles may also be delivered in aerosols. This is made possible by the small size of the nanoparticles.

The nanoparticles of the invention may be formulated as pharmaceutical compositions that may be in the forms of solid or liquid compositions. Such compositions will generally comprise a carrier of some sort, for example a solid carrier or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration, e.g. intraveneously, orally or parenterally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th Edition, 2000, pub. Lippincott, Williams & Wilkins; and Handbook of Pharmaceutical Excipients, 2nd edition, 1994. By way of example, and the compositions are preferably administered to patients in dosages of between about 0.01 and 100 mg of active compound per kg of body weight, and more preferably between about 0.5 and 10 mg/kg of body weight.

The following is presented by way of example and is not to be construed as a limitation to the scope of the claims.

### EXAMPLES

### Example 1

### Synthesis of Nanoparticles

Gold nanoparticles having a corona of carbohydrate ligands or glutathione ligands were synthesised essentially as described previously (WO 2011/154711; and Lund et al., 2011, Biomaterials Vol. 32 pp. 9776-9784, the entire contents of which are expressly incorporated herein by reference).

Oxidized ligand, glutathione (Fluka 49741) was dissolved in 9:1 methanol:water and gold III chloride (Sigma-Aldrich, Poole, UK) added. The organic ligand was used at a fourfold molar excess relative to the gold. The solution was then mixed for 5 min gently on a flat-bed shaker. The nanoparticles were produced by reduction following the rapid addition of a 20 fold molar excess relative to the gold, of freshly made 1 M sodium borohydride (Sigma-Aldrich, Poole, UK) under vigorous vortexing. The samples were vortexed for a total of 30 s followed by a further 1 h gentle mixing on the flat bed shaker. As the nanoparticles are not soluble in methanol/water solvent, initial purification was by bench centrifugation, supernatant removal and dispersion of the nanoparticle pellet in water. Further purification was achieved by 4 water washes in 10 kDa vivaspin centrifugation devices (GE Healthcare). The gold concentration of all nanoparticle preparations was determined by a simple colorimetric assay. Briefly 10 μl of nanoparticle sample or 12 mg/ml gold standard (Fluka (Sigma-Aldrich, Poole, UK)) and blanks were digested with 30 μl of 50:50 water:aqua regia in an ELISA plate for 1 min, this was followed by addition of 150 μl of 2 M NaBr, the 405 nm absorbance was then measured immediately, the assay having excellent linearity over the 0-10 μg range.

### Example 2

### Peptide Binding to Nanoparticles

The present inventors have investigated the ability of the peptide teriparatide to bind nanoparticles.

Teriparatide (marketed under the trade name FORTEO®) is recombinant human parathyroid hormone (1-34), it has an identical sequence to the 34 N-terminal amino acids (the biologically active region) of the 84-amino acid human parathyroid hormone.

Teriparatide has the following sequence: SVSEIQLMHNLGKHLNSMERVEWLRKKLQDVHNF (SEQ ID NO: 1) and a molecular weight of 4118Da and a high pI of 9.8. Given the high pI of this peptide it would have a net positive charge through physiological pHs until pH 9.8 and as such would tend to be electrostatically repulsed by certain nanoparticle (NP) corona compositions which are also cationic at physiological pH. The requirement was for a new NP with ligands that would give the particle a net negative charge at physiological pHs.

Initial NP production experiments focused on using the ligand, 15-Mercapto-4,7,10,13-tetraoxa-pentadecanoic acid (MTPDA), this ligand was costly and only produced NP using a modified alkali based production method, attempts were made to bind teriparatide to this NP indicate that binding may be sub-optimal, albeit some binding had occurred. As a result of these difficulties, attention was turned to glutathione as the NP ligand, this ligand has the advantage of being cheap, it is easily incorporated into NPs, it has a net negative charge at physiological pH's and is also a natural compound found in mammalian cells.

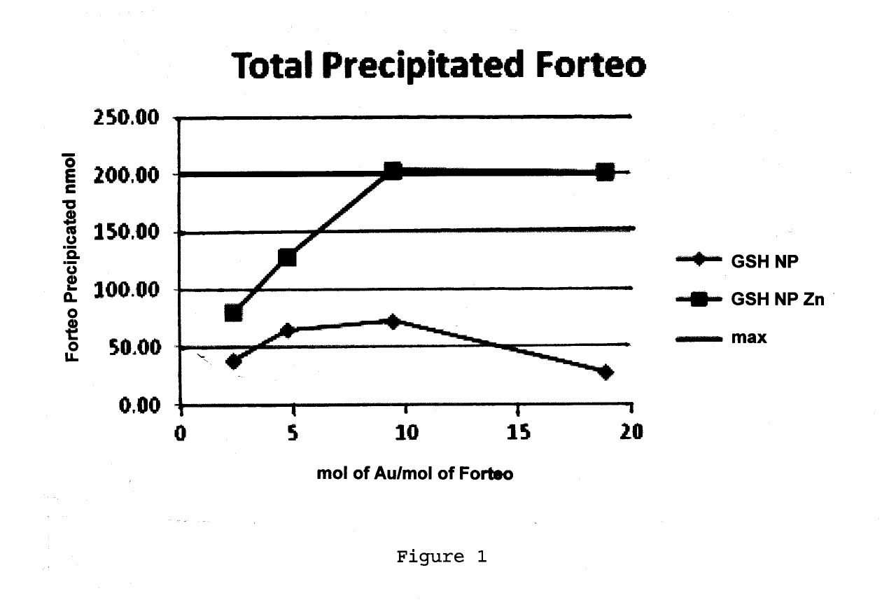

Glutathione nanoparticles (GSHNPs) were found not only to bind teriparatide, but in the presence of Zn²⁺, increased teriparatide binding was apparently achieved (see FIG. 1). The binding assay tested variable amounts of GSHNP against a fixed amount of Forteo, details of the method are given below.

### Teriparatide Binding Method

50 μl (1 mg/ml Au weight NP)+156 μl pH6 Buffer (25 mM KH₂PO₄/NaOH)+222 μl (1 mg/ml teriparatide in H₂O) made up to 750 μl with H₂O. Zn acetate was added at 50 μl of 50 μg/ml when required. The solution was then left to precipitate for 1 hour, centrifuged and the level of remaining teriparatide in the supernatant analysed by BCA assay, this value was then subtracted from the starting level to obtain the bound fraction.

The following considerations regarding binding/precipitation are of relevance. After mixing GSHNP and teriparatide for a fixed time the samples were centrifuged. If a pellet formed that was considered successful binding due to aggregation of complexes with little net charge, it is however, possible that some teriparatide for example could be bound to GSHNP but that this material fails to spin down as such this would then be defined as non-bound.

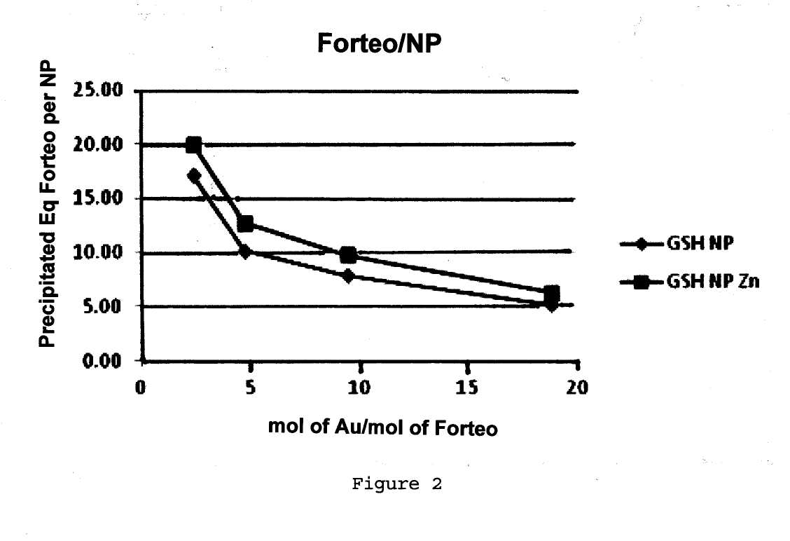

As some NP material was visible in the binding assay supernatants for the test without Zn²⁺ it was possible to quantitate the Gold content and subtract this from the amount expected in the pellet this analysis suggested the effect of Zn²⁺ was not to increase the amount of teriparatide bound to the NP but more likely to aid the co-precipitation of the NPs+teriparatide once formed, as seen in FIG. 2.

The glutathione-based NP successfully bound >15 teriparatide peptide molecules per NP at the highest teriparatide/NP ratio. Zn²⁺ addition also gave unusual data initially suggesting it increased teriparatide binding but it appears most likely that it, in fact, aided precipitation of the teriparatide/NP complex.

The basic binding assay used throughout these following studies was 50 μl teriparatide (at 1.65 or 0.825 mg/ml) in various pH 25 mM potassium phosphate buffers, added to NPs (expressed as Au content) in a total of 200 μl water, test samples mixed and then centrifuged after 30 min and the supernatant assayed for protein content by BCA 560 nm, +/−ve controls included to determine how much material has bound.

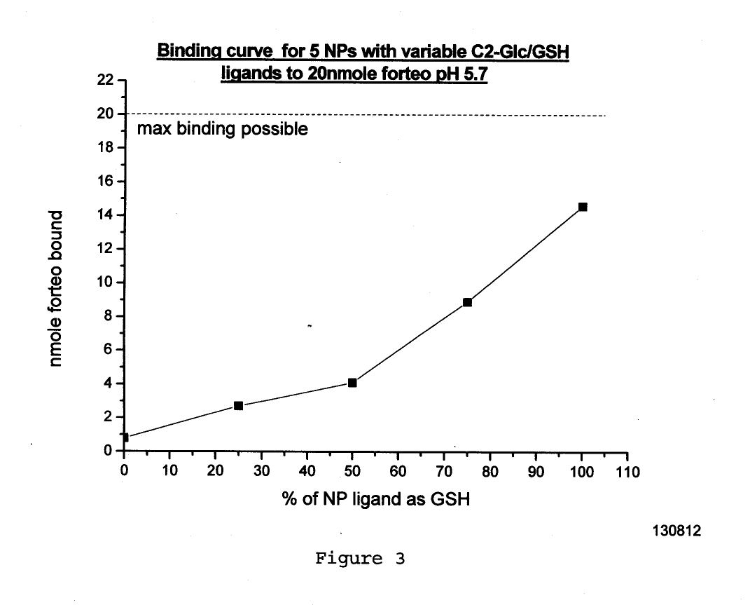

Teriparatide binding to variable ratio C2-Glucose and glutathione (GSH) ligand NPs (in this case 150 nmole Au content used), this was performed at pH 5.7 being approximately midway between the main pKa of GSH carboxyls and the teriparatide pI. The results are shown in FIG. 3.

The data suggest that pure C2GlcNP effectively does not bind teriparatide, and that increased binding of teriparatide occurs as the I of GSH ligand increases, the best binding was observed with the 100% GSHNP (see FIG. 3).

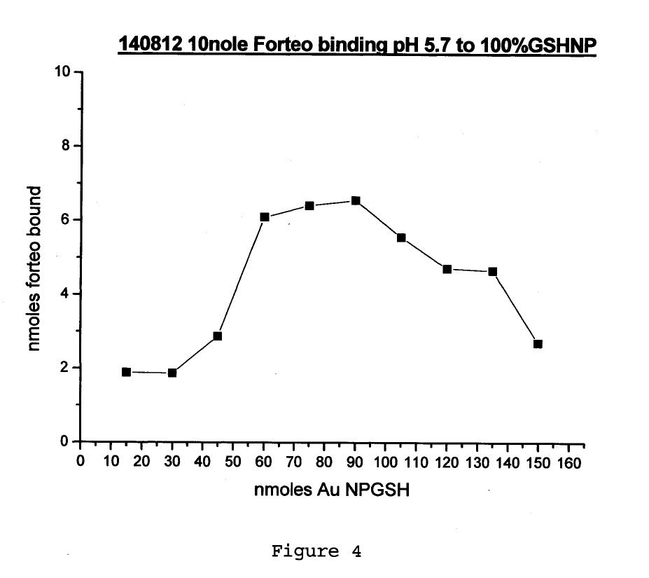

A binding curve was performed with variable/excess GSHNP and a lower level of teriparatide (see FIG. 4).

FIG. 4 shows increased teriparatide binding with increasing NP to 60-90 nmole NP Au, then a steady decrease presumably due to suboptimal binding which prevents NP aggregation/centrifugation. These data suggest a maximal binding of approximately 10 teriparatide molecules per NP (this number assumes 100 Au atoms/NP the 60 nmole Au equates to 0.6 nmole NP which binds 6 nmole teriparatide hence maximum binding seen of 10 teriparatide/NP).

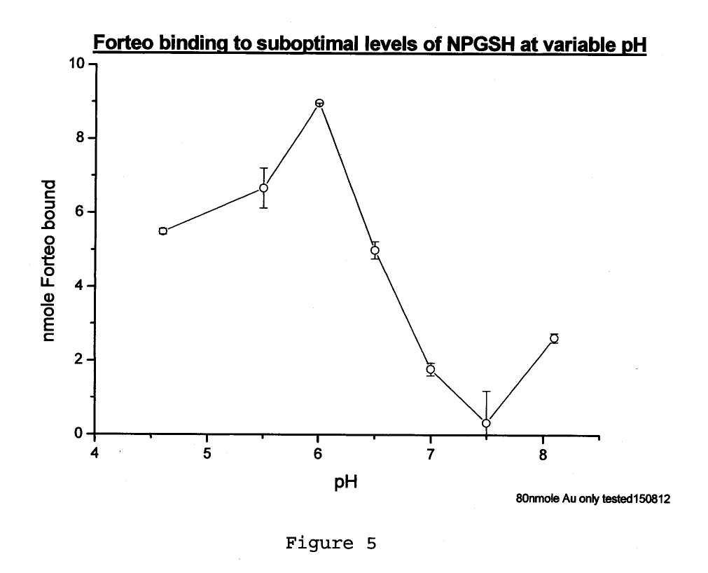

Variable pH binding curves (see FIG. 5).

The effect of pH on binding was tested with variable pH isotonic 25 mM potassium phosphate buffers, the buffer was further diluted 5-fold in the actual binding assay by the aqueous NP component. These data were performed with 100% GSHNP at the 80 nmole Au level only. The data shown in FIG. 5 clearly demonstrate pH binding dependence, the pH around 6 appears ideal; the slight increase in binding at pH 8 is thought to be due to teriparatide precipitation alone as it nears its pI.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A nanoparticle composition comprising:
(a) a nanoparticle comprising:
(i) a core comprising a metal and/or a semiconductor;
(ii) a corona comprising a plurality of ligands covalently linked to the core, wherein said plurality of ligands comprise at least one glutathione; and
(b) at least one teriparatide peptide that is non-covalently bound to the corona.

2. The nanoparticle composition according to claim 1, wherein the teriparatide peptide comprises or consists of:
(i) an amino acid sequence having at least 70%, 80%, 90%, 95% or 99% amino acid sequence identity to the full-length sequence set forth in SEQ ID NO: 1 or 3;
(ii) a peptide comprising or consisting of the full-length amino acid sequence set forth in SEQ ID NO: 1 or 3;
(iii) a peptide comprising or consisting of a variant sequence of the full-length amino acid sequence set forth in SEQ ID NO: 1 or 3, wherein said variant differs by addition, deletion, substitution or modification of not more than 1, 2, 3, 4, 5, 6, 7, 8, 9 or not more than 10 amino acids from said full-length amino acid sequence set forth in SEQ ID NO: 1 or 3;
(iv) a peptide comprising or consisting of a fragment of any one of (i)-(iii), said fragment having a sequence length of at least 15, 20, 25 or 30 amino acids.

3. The nanoparticle composition according to claim 1, wherein the teriparatide peptide exhibits at least 50% of the activity of the teriparatide peptide of SEQ ID NO: 1 or at least 50% of the activity of the teriparatide peptide of SEQ ID NO: 3 in an in vitro or in vivo bioassay of teriparatide activity.

4. The nanoparticle composition according to claim 3, wherein the teriparatide activity is selected from the group consisting of: PTH receptor agonist activity; modification of the osteoblast/osteoclast bone formation/resorption balance; enhancement of kidney calcium and/or magnesium reabsorption; regulation of plasma calcium and/or phosphate concentration; enhancement of conversion of 25(OH) vitamin D3 to 1,25(OH)₂ vitamin D3; and enhancement of intestinal calcium absorption.

5. The nanoparticle composition according to claim 1, wherein the corona comprises at least 5, 10, 20 or at least 50 ligands per core.

6. The nanoparticle composition according to claim 1, wherein the corona comprises at least 5, 10, 20 or at least 50 glutathione ligands per core.

7. The nanoparticle composition according to claim 1, wherein the number of teriparatide peptide molecules bound to the nanoparticle is selected from: 1, 2, 3, 4, 5, 10 or at least 15 per core.

8. The nanoparticle composition according to claim 1, wherein the at least one teriparatide peptide is bound to the corona of the nanoparticle in a reversible manner.

9. The nanoparticle composition according to claim 1, wherein the teriparatide peptide is bound to the corona such that at least a fraction of the bound teriparatide peptide is released from the nanoparticle upon contacting the nanoparticle composition with a physiological solution.

10. The nanoparticle composition according to claim 1, wherein said ligands comprise glutathione alone or in conjunction with other species of ligand.

11. The nanoparticle composition according to claim 10, wherein said ligands comprise combinations of glutathione and carbohydrate ligands.

12. The nanoparticle composition according to claim 1, wherein the nanoparticle composition comprises a plurality of said nanoparticles, wherein at least 10%, 20%, 30%, 40%, 50%, 60%, 701, 80%, 90% or 95% of the nanoparticles in said composition have at least one teriparatide peptide bound.

13. The nanoparticle composition according to claim 1, wherein the nanoparticle composition comprises a carrier in which the nanoparticles and bound teriparatide peptides are suspended.

14. The nanoparticle composition according to claim 13, wherein the composition is in an associated form, a suspension or contained in a single package or container.

15. The nanoparticle composition according to claim 1, wherein the composition is in the form of one or more doses of a defined quantity of teriparatide peptide or of a defined level of teriparatide peptide activity units.

16. The nanoparticle composition according to claim 1, wherein the composition further comprises at least one permeation enhancer that is non-covalently or covalently bound to said core and/or or said corona.

17. The nanoparticle composition according to claim 16, wherein said permeation enhancer is selected from the group consisting of: an alkyl-D-maltoside, tetradecyl-D-maltoside, dodecyl-β-D-maltoside, hexyl-β-D-maltoside, octyl-β-D-maltoside, nonyl-β-D-maltoside, decyl-β-D-maltoside, undecyl-β-D-maltoside, tridecyl-β-D-maltoside, hexadecyl-β-D-maltoside and lysalbinic acid.

18. The nanoparticle composition according to claim 17, wherein said permeation enhancer is non-covalently bound to said corona.

19. A method of treatment of a disorder of bone density in a mammalian subject, the method comprising administering a therapeutically effective amount of a nanoparticle composition of claim 1 to the subject in need of said treatment.

20. A method of increasing bone mineral density in a mammalian subject, the method comprising administering an effective amount of a nanoparticle composition of claim 1 to the subject.

21. The method of claim 19, wherein the subject has, or is at risk of developing, osteoporosis.

22. The method of claim 19, wherein the nanoparticle composition is administered simultaneously, separately or sequentially with one or more therapeutic agents for the control of bone density selected from the group consisting of: a bisphosphonate medication, hormone replacement therapy, calcium, vitamin D; menatetrenone; and vitamin K.

23. The method of claim 19 wherein the nanoparticle composition is administered via a route selected from the group consisting of: intravenous (i.v.), intramuscular (i.m.), intradermal (i.d.), intraperitoneal or subcutaneous (s.c.) injection or infusion; buccal; sublabial; sublingual; by inhalation; via one or more mucosal membranes; urogenital; rectal; intranasal; and dermal.

24. An article of manufacture comprising:
a nanoparticle composition of claim 1;
a container for housing the nanoparticle composition; and
an insert and/or label.

25. A process for producing a nanoparticle composition of claim 1, the process comprising:
providing a nanoparticle comprising a core comprising a metal and/or a semiconductor and a corona comprising a plurality of ligands covalently linked to the core, wherein said plurality of ligands comprise at least one glutathione; and
contacting the nanoparticle with at least one teriparatide peptide under conditions which allow the at least one teriparatide peptide to bind to the corona of the nanoparticle.


SEQUENCE LISTING (3 sequences)

SEQ ID NO: 1 - protein, 34 aa, Homo sapiens
SVSEIQLMHNLGKHLNSMERVEWLRKKLQDVHNF

SEQ ID NO: 2 - protein, 115 aa, Homo sapiens
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 3 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ